(19) United States
(12) Patent Application Publication
(10) Pub. No.: US 2016/0324531 A1
(43) Pub. Date: Nov. 10, 2016

(54) SINUS TREATMENT

(71) Applicant: Rainbow Medical Ltd., Herzliya (IL)

(72) Inventors: Yossi Gross, Moshav Mazor (IL); Hadar Better, Tel Aviv (IL)

(21) Appl. No.: 14/704,841

(22) Filed: May 5, 2015

(51) Int. Cl. A61B 17/16; A61B 17/3205; A61B 17/3209; A61B 17/24

(52) U.S. Cl. CPC A61B 17/1688; A61B 17/24; A61B 17/32053; A61B 17/3209; A61B 2017/320056

(57) ABSTRACT

Apparatus and methods are described, including a method for opening a natural ostium of a sinus of a subject. An artificial hole is formed in a bone wall of the sinus, and a stent is implanted in the hole. The ostium is then opened by pumping a fluid through the stent such that the fluid is in direct contact with the sinus. Other applications are also described.

### CROSS REFERENCE TO RELATED APPLICATIONS

The present application is related to an application entitled “Sinus Treatment” to Gross et al., filed on even date herewith.

### FIELD OF THE INVENTION

Applications of the present invention relate generally to treatment of sinus disorders, and specifically, to apparatus and methods for opening a natural ostium of a sinus.

### BACKGROUND

Sinusitis is a common condition, characterized by symptoms such as nasal discharge, facial and ear pressure and pain, headache, loss of smell, fever, cough and fatigue. Physiological clearing of paranasal sinuses is via mucociliary transport through the ostia (natural sinus openings into a nasal cavity). Disruption of this function, often associated with partial blockage of an ostium, allows stagnation of mucous secretions and alteration of pH and other physiologic parameters, making the sinus conditions more favorable to microbiological growth and, therefore, susceptible to sinusitis. Treatment of chronic sinusitis typically includes antibiotics, steroids, decongestants and, in some cases, surgical procedures such as lavage.

US 2013/0030545 to Gross et al., which is incorporated herein by reference, describes paranasal sinus apparatus, including a hole-forming member configured to form a hole through a bone wall of a paranasal sinus of a subject. An implant is provided, having a proximal end and a distal end, and which is shaped to define a lumen. The implant is couplable to the hole-forming member, and is securable to the wall and extendable through the hole, such that the proximal end is disposed external to the paranasal sinus and the distal end is disposed within the paranasal sinus. The implant includes a biodegradable material.

### SUMMARY OF THE INVENTION

Applications of the present invention include a method for opening a natural ostium of a sinus of a subject. An artificial hole is formed in a bone wall of the sinus, and a stent is implanted in the hole. A fluid is then pumped through the stent, until the fluid pressure inside the sinus increases sufficiently to force the ostium open.

Applications of the present invention also include apparatus for boring the artificial hole in the bone wall of the sinus. The apparatus comprises a boring element, typically comprising a drill bit and/or a punch. The boring element has an outer surface that is (a) electrically conductive at a distal end of the boring element, and (b) is not electrically conductive at a portion of the boring element that is proximal to the distal end, around a full circumference of the boring element. A drill is configured to rotate the boring element around a central longitudinal axis of the boring element, thus facilitating the boring of the hole. Prior to boring the hole, the distal end of the boring element is used to apply a radiofrequency (RF) electrical signal to soft tissue that covers the bone wall of the sinus, in order to cauterize the tissue. Since the boring element is electrically conductive only at its distal end, the applied RF signal generally does not damage tissue in the nasal cavity or skin adjacent to the nasal cavity.

There is therefore provided, in accordance with some applications of the present invention, a method for opening a natural ostium of a sinus of a subject, the method including:

forming an artificial hole in a bone wall of the sinus;

implanting a stent in the hole; and

opening the ostium by pumping a fluid through the stent such that the fluid is in direct contact with the sinus.

In some applications, pumping the fluid includes pumping an agent selected from the group consisting of: an antibiotic agent, and an antibiofilm agent.

In some applications, pumping the fluid includes pulsatingly pumping the fluid.

In some applications, pumping the fluid includes pumping a fluid having a temperature greater than 40 degrees.

In some applications, the method further includes directing a stream of fluid at the ostium.

In some applications, the method further includes, following the opening of the ostium, rinsing the sinus by alternatingly pumping a rinsing fluid into and out of the sinus.

In some applications, the method further includes using an endoscope to view the ostium while pumping the fluid.

In some applications, the method further includes stopping to pump the fluid in response to viewing, using the endoscope, that the ostium is open.

In some applications, the method further includes stopping to pump the fluid in response to the fluid exiting a nose of the subject.

In some applications, the method further includes detecting a pressure of the fluid while pumping the fluid.

In some applications, the method further includes stopping to pump the fluid in response to detecting a decrease in the pressure of the fluid.

In some applications, detecting the pressure includes using a pressure sensor to detect the pressure.

There is further provided, in accordance with some applications of the present invention, a method for rinsing a sinus of a subject, the method including:

forming an artificial hole in a bone wall of the sinus; and

subsequently to forming the artificial hole, rinsing the sinus by passing a fluid into the sinus through a natural ostium of the sinus.

There is further provided, in accordance with some applications of the present invention, apparatus for boring an artificial hole in a bone wall of a sinus, the apparatus including:

a boring element having an outer surface that is:
electrically conductive at a distal end of the boring element, and
not electrically conductive at a portion of the boring element that is proximal to the distal end of the boring element, around a full circumference of the boring element; and
a drill configured to rotate the boring element around a central longitudinal axis of the boring element.

In some applications,

the distal end of the boring element includes an electrically conductive material,

the portion of the boring element that is proximal to the distal end includes an electrically insulative material shaped to define a boring-element lumen, and

the boring element includes an electrically-conductive element disposed within the boring-element lumen and coupled to the electrically conductive material.

In some applications, the portion of the boring element that is proximal to the distal end includes an electrically insulative coating.

In some applications, the drill is configured to rotate the boring element around the central longitudinal axis of the boring element at a steady-state speed of 100-1000 rotations per minute.

In some applications, the drill is configured to rotate the boring element around the central longitudinal axis of the boring element at a steady-state speed of 100-500 rotations per minute.

In some applications, the boring element includes a drill bit.

In some applications, the apparatus further includes a punch shaped to define a punch lumen,

the drill is configured to rotate the punch around a central longitudinal axis of the punch, and

the drill bit is disposed within the punch lumen.

In some applications, the punch has an outer surface that is (a) electrically conductive at a distal end of the punch, and (b) is not electrically conductive at a portion of the punch that is proximal to the distal end of the punch, around a full circumference of the punch.

In some applications, the drill bit protrudes 1-4 mm from a distal end of the punch.

In some applications, the drill bit protrudes 2-3 mm from a distal end of the punch.

In some applications, the boring element includes a punch.

In some applications, the apparatus further includes a drill bit disposed within a lumen of the punch.

In some applications, the drill includes a controller configured to, upon being activated by a user exactly one time:

apply a radiofrequency electrical signal to tissue that covers the bone wall, via the boring element, and

subsequently to beginning to apply the radiofrequency signal, initiate rotating the boring element.

In some applications, the controller is configured to initiate rotating the boring element 1-5 seconds after beginning to apply the radiofrequency signal.

In some applications, the drill includes a controller configured to:

upon being activated by a user a first time, apply a radiofrequency electrical signal to tissue that covers the bone wall, via the boring element, and

upon being activated by the user a second time following the first time, initiate rotating the boring element.

In some applications, the drill includes a force sensor configured to sense a force applied to the boring element, the drill being configured to initiate rotating the boring element in response to a signal from the force sensor indicating that a force is being applied to the boring element by the bone wall.

There is further provided, in accordance with some applications of the present invention, a method for drilling an artificial hole in a bone wall of a sinus, the method including:

applying a radiofrequency electrical signal to soft tissue via a non-rotating boring element; and

subsequently, drilling the artificial hole in the bone wall, by using a drill to rotate the boring element around a central longitudinal axis of the boring element.

In some applications,

the drill includes a controller,

applying the radiofrequency electrical signal includes applying the radiofrequency electrical signal by activating the controller a first time, and

drilling the artificial hole in the bone wall includes initiating rotation of the boring element by activating the controller a second time.

In some applications,

the drill includes a controller, and the method includes, by activating the controller exactly one time:

applying the radiofrequency electrical signal, and subsequently, initiating rotation of the boring element.

In some applications, rotating the boring element includes rotating the boring element at a steady-state speed of 100-1000 rotations per minute.

In some applications, rotating the boring element includes rotating the boring element at a steady-state speed of 100-500 rotations per minute.

In some applications, using the drill to rotate the boring element includes beginning to rotate the boring element 1-5 seconds after beginning to apply the radiofrequency electrical signal.

In some applications, the method further includes using a force sensor to sense a force applied to the boring element by the bone wall, and using the drill to rotate the boring element includes beginning to rotate the boring element in response to the sensed force.

The present invention will be more fully understood from the following detailed description of applications thereof, taken together with the drawings, in which:

### BRIEF DESCRIPTION OF THE DRAWINGS

FIG. 1 is a schematic illustration of a method for opening a natural ostium of a sinus of a subject, in accordance with some applications of the present invention;

FIGS. 2A-B are schematic illustrations of apparatus for boring an artificial hole in a bone wall of a sinus, in accordance with some applications of the present invention; and

FIG. 3 is a schematic illustration of a technique for boring an artificial hole in a bone wall of a sinus, in accordance with some applications of the present invention.

### DETAILED DESCRIPTION OF APPLICATIONS

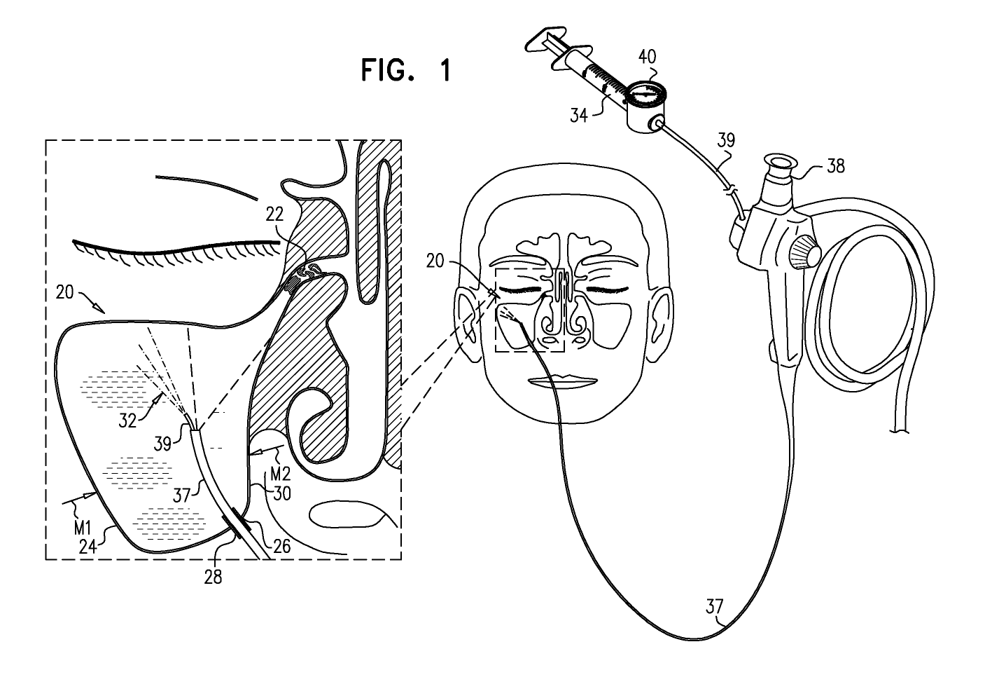

Reference is now made to FIG. 1, which is a schematic illustration of a method 20 for opening a natural ostium 22 of a sinus 24 of a subject, in accordance with some applications of the present invention. FIG. 1 shows a blocked ostium 22. To open the ostium, an artificial hole 26 is formed in a bone wall 30 of sinus 24, a stent 28 is implanted in hole 26, and a fluid 32 (e.g., air or saline) is pumped through the stent. As shown in FIG. 1, the fluid is pumped through the stent such that the fluid is in direct contact with the sinus, i.e., the fluid is not pumped into a balloon or other artificial receptacle. It is noted that the scope of the present invention includes forming hole 26, and implanting stent 28, at any one of various sites along bone wall 30. For example, markers M1 and M2 in FIG. 1 point to two sites that may be chosen, alternatively to the implantation site shown in the figure.

In some applications, as shown in FIG. 1, a fluid-delivery tube 39 is passed through stent 28, and the fluid is pumped through the stent by being pumped through the lumen of tube 39. In other applications, the fluid is pumped directly through the stent. For example, a distal end of a tube may be coupled to the proximal opening of the stent, and the fluid may be pumped through the stent via the tube.

Typically, fluid 32 is pumped into the sinus until the fluid pressure inside the sinus increases sufficiently to force the ostium open. In some applications, e.g., when tube 39 is passed into the sinus, the ostium is opened by directing a stream of fluid at the ostium.

In some applications, as shown in FIG. 1, a syringe 34 is used to pump the fluid. Alternatively or additionally, another type of mechanical pump, and/or an electric pump, may be used. In some applications, to facilitate the opening of the ostium using less fluid than would otherwise be necessary, the fluid is pulsatingly pumped.

In some applications, an antibiotic agent and/or an antibiofilm agent is pumped through the stent. For example, fluid 32 may contain a mix of saline and antibiotic, and/or the antibiotic may be pumped into the sinus following the pumping of the saline. In some applications, fluid 32 has a temperature greater than 40 degrees. The relatively high temperature of the fluid helps soften any mucus that may be blocking the ostium.

In some applications, an endoscope 38 is used to view the ostium while pumping the fluid. (In such applications, as shown in FIG. 1, fluid-delivery tube 39 may run through a working channel of an endoscope tube 37 of endoscope 38.) In such applications, the physician may stop pumping the fluid in response to viewing, using the endoscope, that the ostium is open. In some applications, the pressure of the fluid is detected while pumping the fluid, e.g., using a pressure sensor 40, and/or by the physician feeling the pressure against the plunger of the syringe. In such applications, the physician may stop pumping the fluid in response to detecting a decrease in the pressure of the fluid, since the decrease in pressure indicates that the ostium was opened. Alternatively or additionally to the above, the physician may stop pumping the fluid in response to the fluid exiting the subject's nose, since the exit of the fluid from the nose indicates that the ostium was opened.

Typically, following the opening of the ostium, the sinus is rinsed. In some applications, a rinsing fluid (e.g., saline) is alternatingly pumped into and out of the sinus, via artificial hole 26 and/or natural ostium 22. Alternatively or additionally, rinsing fluid may be passed into the sinus through the natural ostium, such that the rinsing fluid exits the sinus through the artificial hole.

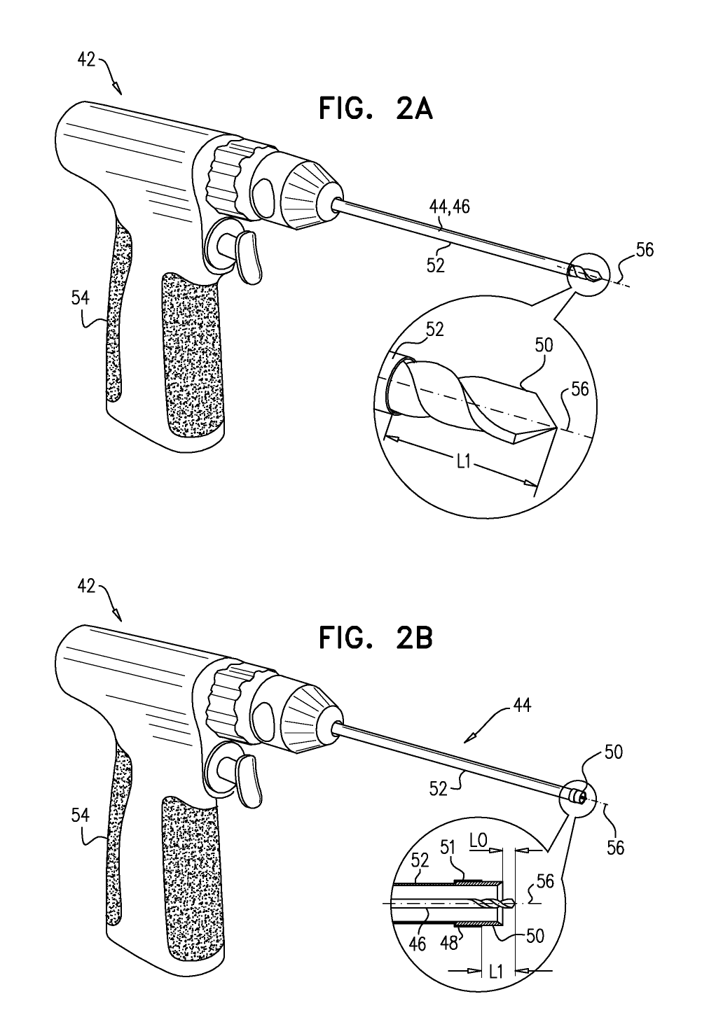

Reference is now made to FIGS. 2A-B, which are schematic illustrations of apparatus 42 for boring artificial hole 26 (FIG. 1), in accordance with some applications of the present invention. (It is noted that throughout the claims and specification of the present application, the terms “boring” and “drilling” may be used interchangeably. For example, a method or apparatus for “drilling” an artificial hole may be described as a method or apparatus for “boring” an artificial hole, and vice versa.) Apparatus 42 comprises a boring element 44, typically comprising a drill bit 46 and/or a punch 48.

Boring element 44 has an outer surface that is (a) electrically conductive at a distal end 50 of the boring element, and (b) is not electrically conductive at a portion 52 of the boring element that is proximal to the distal end, around a full circumference of the boring element. A drill is configured to rotate the boring element around a central longitudinal axis 56 of the boring element, thus facilitating the boring of the hole. (A drill is a tool having a cutting tool, usually a drill bit or driver bit, which is used for boring holes in various materials. The cutting tool may be permanently fixed to the drill, or removeably attachable to the drill. As used in the present application, including in the claims, a “central longitudinal axis” of an elongate structure is the set of all centroids of cross-sectional sections of the structure along the structure.)

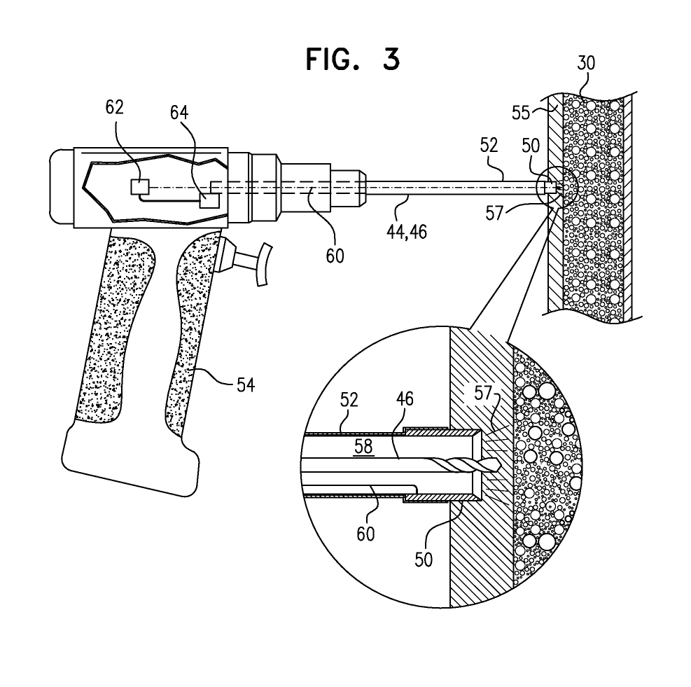

Typically, the length of electrically-conductive distal end 50 is relatively small, relative to the total length of the boring element. (For example, the length L1 of distal end 50 may be at least 1 mm and/or less than 5 mm.) As described hereinbelow with reference to FIG. 3, the distal end of the boring element is used to apply a radiofrequency (RF) electrical signal to soft tissue that covers the bone wall of the sinus, in order to cauterize the tissue. The relatively small length of electrically-conductive distal end 50 helps keep the applied RF signal from inadvertently damaging tissue in the nasal cavity or skin adjacent to the nasal cavity.

Reference is now made to FIG. 3, which is a schematic illustration of a technique for boring artificial hole 26 (FIG. 1), in accordance with some applications of the present invention. Before boring the artificial hole in the bone wall of the sinus, the boring element is brought into contact with soft tissue 55 that covers the outside of the bone wall. Without rotating the boring element, an RF electrical signal 57 is applied to the soft tissue, via the boring element. Subsequently, the artificial hole is drilled in the bone wall, by using the drill to rotate the boring element around central longitudinal axis 56 (FIGS. 2A-B). The application of RF electrical signal 57 helps reduce the amount of bleeding that occurs prior to the hole being drilled. It is noted that the far side of the bone wall is also covered by a layer of soft tissue. However, this layer is relatively thin, such that relatively little bleeding occurs as the boring element passes through the far-side tissue and into the sinus.

Typically, drill 54 comprises a controller 62, comprising, for example, control circuitry. In general, it is desired not to begin rotating the boring element until after the application of the RF signal to tissue 55 has begun, and until after the boring element has come into contact with the bone wall. (The boring element may typically be passed easily through the tissue, without a need to rotate the boring element.) The scope of the present invention includes several techniques for addressing this objective, one or more of these techniques typically being facilitated by controller 62, as follows:

(i) In some applications, controller 62 is configured to, upon being activated by a user exactly one time, (a) begin to apply RF signal 57 to tissue 55 via the boring element, as described hereinabove, and (b) subsequently to beginning to apply the RF signal, initiate rotating the boring element. For example, the controller may initiate rotating the boring element at least one second and/or less than five seconds after beginning to apply the RF signal. In such applications, the user will typically (a) begin to apply the RF signal by activating the controller, (b) approximately simultaneously with, or subsequently to, beginning to apply the RF signal, push the boring element through the tissue, the RF signal generally preventing significant bleeding from occurring as the boring element is pushed through the tissue, and (c) upon bringing the boring element into contact with the bone wall, wait a short period of time, until the boring automatically begins.

(ii) In other applications, the user activates the controller twice. Upon the first activation, the controller begins to apply the RF signal to tissue 55. Upon the subsequent second activation, the controller initiates rotating the boring element. In such applications, the user will typically (a) begin to apply the RF signal by activating the controller a first time, (b) approximately simultaneously with, or subsequently to, beginning to apply the RF signal, push the boring element through the tissue, the RF signal generally preventing significant bleeding from occurring as the boring element is pushed through the tissue, and (c) upon bringing the boring element into contact with the bone wall, initiate the boring by activating the controller a second time.

(iii) In yet other applications, the drill comprises a force sensor 64 configured to sense a force applied to the boring element, and the drill is configured to initiate rotating the boring element in response to a signal from the force sensor indicating that a force is being applied to the boring element by the bone wall. (For example, force sensor 64 may communicate such a signal wiredly or wirelessly to controller 62, which then initiates the rotation of the boring element in response to the signal.) In such applications, the user will typically (a) begin to apply the RF signal by activating the controller, (b) approximately simultaneously with, or subsequently to, beginning to apply the RF signal, push the boring element through the tissue, the RF signal generally preventing significant bleeding from occurring as the boring element is pushed through the tissue, and (c) press the boring element against the bone wall, thus “triggering” the force sensor and initiating the boring.

In some applications, the RF signal is terminated after a certain predetermined period of time has transpired, at the time that the drilling begins, or at some other time before the end of the drilling procedure. In other applications, the RF signal is applied for the entire duration of the drilling procedure.

While the outer surface of distal end 50 of the boring element comprises an electrically conductive material, such as a metal, the outer surface of portion 52 comprises an electrically insulative material, such as a plastic. For example, the outer surface of portion 52 may comprise an electrically insulative coating. For example, FIG. 2B shows an electrically insulative coating 51 covering all of punch 48, except for the distal end of the punch. As noted above, the electrical non-conductivity of portion 52 helps keep the applied RF signal from inadvertently damaging tissue in and around the nasal cavity.

In some applications, the interior of the boring element is electrically conductive, and the RF signal is delivered to the distal end of the boring element via the electrically-conductive interior of the boring element. Alternatively or additionally, boring element 44 comprises an electrically-conductive element 60, e.g., a wire, that delivers the RF signal to the distal end of the boring element. In some applications, portion 52 is shaped to define a boring-element lumen 58. Electrically-conductive element 60 is disposed within boring-element lumen 58, and is coupled to the electrically conductive material at distal end 50.

The application of the RF signal may be unipolar or bipolar. That is, a second electrode may be disposed outside the body of the patient or within the body of the patient, e.g., on the apparatus itself (as described, for example, hereinbelow with reference to FIG. 2B). As the RF signal passes between the proximal end of the boring element (which acts as a first electrode) and the second electrode, soft tissue 55 is cauterized.

Typically, drill 54 is configured to rotate the boring element around the central longitudinal axis of the boring element at a steady-state speed of at least 100 and/or less than 1000 rotations per minute (e.g., 100-500 rotations per minute), which is the steady-state speed that is typically used for boring through the bone wall. In some applications, drill 54 is configured to rotate the boring element alternatingly clockwise and counterclockwise.

Reference is again made to FIG. 2B. In some applications, apparatus 42 comprises both drill bit 46 and punch 48. Typically, in such applications, the drill bit is disposed within the lumen of the punch. For example, the drill bit may be centered within the punch lumen, such that central longitudinal axis 56 is the central longitudinal axis of both the drill bit and the punch. The drill rotates both the drill bit and the punch around central longitudinal axis 56, typically together at the same rotation speed. Typically, the drill bit protrudes a distance L0 of at least 1 and/or less than 4 mm, e.g., 2-3 mm, from the distal end of the punch. As the drill bit begins to drill into the bone wall, the drill bit centers the punch over the desired site for the artificial hole, and stabilizes the drill. The punch then bores a hole through the bone wall, the boring of the punch being facilitated by the stabilizing of the drill.

In some applications, each of the drill bit and the punch has (i) an electrically-conductive distal end, and (ii) a non-electrically conductive portion that is proximal to the distal end, the non-electrically conductive portion being non-electrically conductive around the full circumference of the drill bit/punch. In such applications, the RF signal is typically passed between the respective distal ends of the drill bit and punch. In other applications, only the drill bit, or only the punch, has an electrically-conductive distal end, while the other boring element is generally non-electrically conductive, even at its distal end. In such applications, the RF signal may be passed between (i) the electrically-conductive distal end, and (ii) a second electrode located inside or outside the body of the subject. In some applications, the distal end of punch 48 is divided into two electrically-conductive “half-tube” portions that are electrically isolated from one another by an electrically-insulative material. (Thus, for example, a transverse cross-section of the distal end of the punch may have electrically-insulative material at the “12:00” and “6:00” points of the cross-section, and may be otherwise electrically-conductive.) In such applications, the RF signal may be passed between the two electrically-conductive portions.

In some applications, laser and/or ultrasound drilling is used to bore through the bone wall of the sinus.

In some applications, a suction tube and/or an irrigation tube run alongside the boring element, and/or longitudinally through the boring element. (For example, with reference to FIG. 3, a suction tube and/or an irrigation tube may run alongside drill bit 46, though boring-element lumen 58.) The suction tube and/or irrigation tube facilitate the boring of the hole in the wall of the sinus.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-13. (canceled)

14. Apparatus for boring an artificial hole in a bone wall of a sinus, the apparatus comprising:
a boring element having an outer surface that is:
electrically conductive at a distal end of the boring element, and
not electrically conductive at a portion of the boring element that is proximal to the distal end of the boring element, around a full circumference of the boring element; and
a drill configured to rotate the boring element around a central longitudinal axis of the boring element.

15. The apparatus according to claim 14,
wherein the distal end of the boring element comprises an electrically conductive material,
wherein the portion of the boring element that is proximal to the distal end comprises an electrically insulative material shaped to define a boring-element lumen, and
wherein the boring element comprises an electrically-conductive element disposed within the boring-element lumen and coupled to the electrically conductive material.

16. The apparatus according to claim 14, wherein the portion of the boring element that is proximal to the distal end comprises an electrically insulative coating.

17. The apparatus according to claim 14, wherein the drill is configured to rotate the boring element around the central longitudinal axis of the boring element at a steady-state speed of 100-1000 rotations per minute.

18. The apparatus according to claim 17, wherein the drill is configured to rotate the boring element around the central longitudinal axis of the boring element at a steady-state speed of 100-500 rotations per minute.

19. The apparatus according to claim 14, wherein the boring element comprises a drill bit.

20. The apparatus according to claim 19, further comprising a punch shaped to define a punch lumen,
wherein the drill is configured to rotate the punch around a central longitudinal axis of the punch, and
wherein the drill bit is disposed within the punch lumen.

21. The apparatus according to claim 20, wherein the punch has an outer surface that is (a) electrically conductive at a distal end of the punch, and (b) is not electrically conductive at a portion of the punch that is proximal to the distal end of the punch, around a full circumference of the punch.

22. The apparatus according to claim 20, wherein the drill bit protrudes 1-4 mm from a distal end of the punch.

23. The apparatus according to claim 22, wherein the drill bit protrudes 2-3 mm from the distal end of the punch.

24. The apparatus according to claim 14, wherein the boring element comprises a punch.

25. The apparatus according to claim 24, further comprising a drill bit disposed within a lumen of the punch.

26. The apparatus according to claim 14, wherein the drill comprises a controller configured to, upon being activated by a user exactly one time:
apply a radiofrequency electrical signal to tissue that covers the bone wall, via the boring element, and
subsequently to beginning to apply the radiofrequency signal, initiate rotating the boring element.

27. The apparatus according to claim 26, wherein the controller is configured to initiate rotating the boring element 1-5 seconds after beginning to apply the radiofrequency signal.

28. The apparatus according to claim 14, wherein the drill comprises a controller configured to:
upon being activated by a user a first time, apply a radiofrequency electrical signal to tissue that covers the bone wall, via the boring element, and
upon being activated by the user a second time following the first time, initiate rotating the boring element.

29. The apparatus according to claim 14, wherein the drill comprises a force sensor configured to sense a force applied to the boring element, the drill being configured to initiate rotating the boring element in response to a signal from the force sensor indicating that a force is being applied to the boring element by the bone wall.

30. A method for drilling an artificial hole in a bone wall of a sinus, the method comprising:
applying a radiofrequency electrical signal to soft tissue via a non-rotating boring element; and
subsequently, drilling the artificial hole in the bone wall, by using a drill to rotate the boring element around a central longitudinal axis of the boring element.

31. The method according to claim 30,
wherein the drill includes a controller,
wherein applying the radiofrequency electrical signal comprises applying the radiofrequency electrical signal by activating the controller a first time, and
wherein drilling the artificial hole in the bone wall comprises initiating rotation of the boring element by activating the controller a second time.

32. The method according to claim 30,
wherein the drill includes a controller, and wherein the method comprises, by activating the controller exactly one time:
applying the radiofrequency electrical signal, and
subsequently, initiating rotation of the boring element.

33. The method according to claim 30, wherein rotating the boring element comprises rotating the boring element at a steady-state speed of 100-1000 rotations per minute.

34. The method according to claim 33, wherein rotating the boring element comprises rotating the boring element at a steady-state speed of 100-500 rotations per minute.

35. The method according to claim 30, wherein using the drill to rotate the boring element comprises beginning to rotate the boring element 1-5 seconds after beginning to apply the radiofrequency electrical signal.

36. The method according to claim 30, further comprising using a force sensor to sense a force applied to the boring element by the bone wall, wherein using the drill to rotate the boring element comprises beginning to rotate the boring element in response to the sensed force.
